(19) United States
(12) Patent Application Publication
(10) Pub. No.: US 2005/0178849 A1
(43) Pub. Date: Aug. 18, 2005

(54) Air-O-Matic

(76) Inventor: Gregory Harris, Shreveport, LA (US)

Correspondence Address: Gregory Harris, 6414 Canal Blvd., Shreveport, LA, 71108, US

(21) Appl. No.: 10/776,276

(22) Filed: Feb. 12, 2004

(51) Int. Cl. A61L 9/04

(52) U.S. Cl. 239/34

(57) ABSTRACT

The Air-O-Matic consists of a push button located inside the vehicle, which is connected by at tube that leads to a reservoir located in the hood of the vehicle. The reservoir contains a replacible cartridge.

### BACKGROUND OF THE INVENTION

The invention pertains to the automobile industry. The invention resolves the problem of odors that can be found inside vehicles.

### BRIEF SUMMARY OF THE INVENTION

The invention disburses fragrance throughout the vehicle, ridding the vehicle of odors. The invention dispenses with the need to place hanging air fresheners or canisters placed under the seats of vehicles. The invention is easily installable in new or used automobiles.

### BRIEF DESCRIPTION OF THE SEVERAL VIEWS OF THE DRAWING

FIG. 1A depicts the location of the push button which releases the fragrance inside the vehicle.

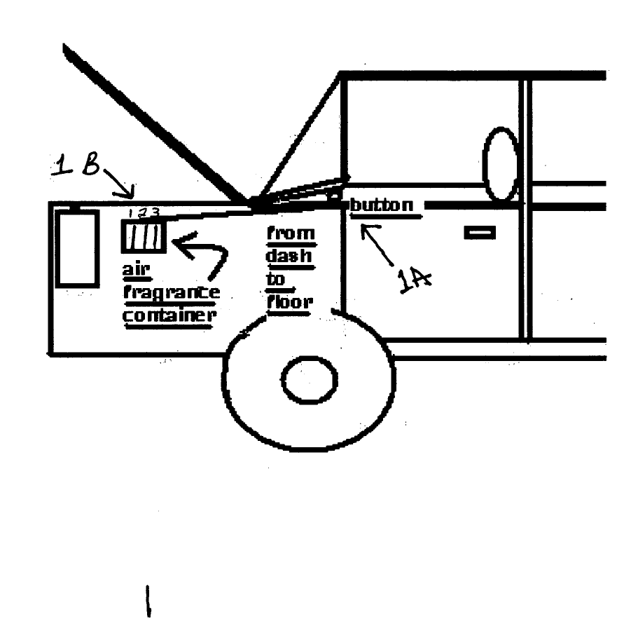

FIG. 1B depicts the location of the reservoir and cartridge that contains the fragrance.

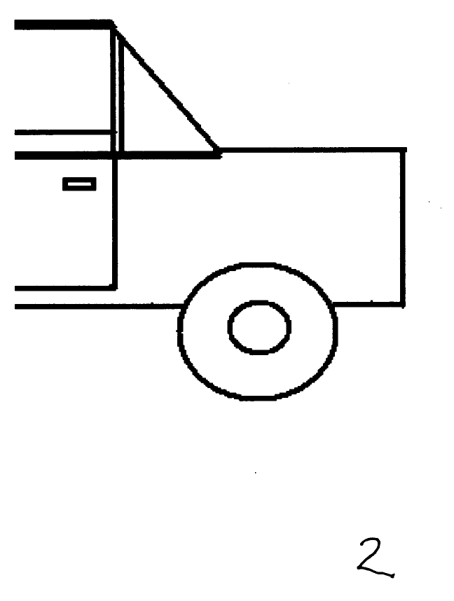

### DETAILED DESCRIPTION OF THE INVENTION

The push button, located inside the vehicle, releases fragrance throughout the vehicle. The fragrance is contained in a reservoir which is located under the hood of the vehicle. Alternatively, the reservoir can also be placed in the trunk of the vehicle. Cartridges containing various fragrances are placed in the reservoir.

1. What I claim as my invention is an automobile air freshener which is operated by pressing a push button which releases fragrance inside the vehicle.
